(19) United States
(12) Patent Application Publication
(10) Pub. No.: US 2026/0263273 A1
(43) Pub. Date: Sep. 10, 2026

(54) GLAUCOMA DRAINAGE DEVICE WITH EXPANDABLE ANCHOR

(71) Applicant: InnFocus, Inc., Miami, FL (US)

(72) Inventors: Leonard Pinchuk, Miami, FL (US); Yasushi Pedro Kato, Weston, FL (US); Anh Le, Miami, FL (US)

(73) Assignee: InnFocus, Inc., Miami, FL (US)

(21) Appl. No.: 19/165,919

(22) PCT Filed: Feb. 23, 2024
(86) PCT No.: PCT/US2024/017130

§ 371 Date: Sep. 16, 2025

Related U.S. Application Data
(60) Provisional application No. 63/454,006, filed on Mar. 22, 2023.

(51) Int. Cl. A61F 9/007

(52) U.S. Cl. CPC A61F 9/00781

(57) ABSTRACT

A glaucoma drainage device is provided having an elongate body that drains aqueous humor from the anterior chamber of the eye. At least one anchor or wire is configured to extend radially outward beyond the body for fixing the body in the eye. The device including the anchor or wire can be housed within the distal end of a needle inserter and deployed therefrom to implant the device into the eye. The deployment can be carried out either ab externo or ab interno. When the device is loaded inside the needle inserter, the wire or anchor (or part thereof) is retained in a compressed state. When deployed from the needle inserter into the eye, the wire or anchor (or part thereof) is configured to automatically self-expand relative to the compressed state and fixate the device in the eye. Related systems and methods of treating glaucoma are also described and claimed.

### CROSS-REFERENCE TO RELATED APPLICATION(S)

The present disclosure claims priority from U.S. Provisional Appl. No. 63/454,006, filed on Mar. 22, 2023, entitled “GLAUCOMA DRAINAGE DEVICE WITH EXPANDABLE ANCHOR” Attorney Docket No. INN-065P, herein incorporated by reference in its entirety.

### BACKGROUND

### 1. Field

The present disclosure relates to devices that shunt or drain aqueous humor from the anterior chamber of the eye.

### 2. State of the Art

Aqueous humor is produced by the eye's ciliary body and flows from the ciliary body into the anterior chamber, out through a spongy tissue at the front of the eye called the trabecular meshwork and into a drainage canal. In a healthy eye, continuous drainage of aqueous humor keeps intraocular pressure at a normal level. However, in most types of glaucoma, proper circulation of aqueous humor is disrupted, causing the level of intraocular pressure to be elevated. In open-angle glaucoma, fluid does not flow freely through the trabecular meshwork, causing an increase in intraocular pressure, damage to the optic nerve and vision loss. Reduction of intraocular pressure is a means of stopping the progression of optic nerve damage, which if untreated can lead to blindness. The risk factors for the development of glaucoma include elevated intraocular pressure (IOP), age, ethnicity, positive finding for the condition in the family history, and thin central corneal thickness. However, IOP remains the only risk factor readily amenable to therapy.

Management of IOP is the mainstay of glaucoma treatment. Reduction of IOP by pharmaceutical, laser or surgical means has long been the standard treatment for glaucoma. In current practice, initial treatment begins with medical or laser therapy; however, incisional surgery can be performed if medications or laser therapy do not satisfactorily reduce IOP and/or prevent visual field progression. Such incisional surgery can include trabeculectomy or implantation of a glaucoma drainage device (GDD) or a MIGS device that shunt or drain aqueous humor from the anterior chamber of the eye. MIGS is a new type of glaucoma drainage device category, which is an abbreviation for “Minimally Invasive Glaucoma Surgery” which was coined in about 2012. MIGS describes several new devices used to shunt aqueous humor to space under the conjunctiva and Tenon's capsule, or to suprachoroidal space, or through the trabecular meshwork into Schlemm's canal. Some MIGS devices are implanted into the eye using an ab externo procedure where the glaucoma drainage device is inserted from outside the eye through a tissue passageway leading to the anterior chamber of the eye.

Other MIGS devices are implanted into the eye using an ab interno procedure where the glaucoma drainage device is inserted from a position inside the anterior chamber through a tissue passageway leading to space outside the anterior chamber. Two important MIGS devices that shunt aqueous humor to space under the conjunctiva and Tenon's capsule are the PRESERFLO® MicroShunt (Santen, Osaka, Japan) and XEN Gel Stent (Allergan (Dublin, Ireland).

The XEN Gel Stent is a tube made from crosslinked gelatin, which is indicated to be injected from inside the eye (ab interno) through the trabecular meshwork and under the limbus, terminating under the conjunctiva and Tenon's capsule where it forms a small blister called a bleb. A problem with the XEN Gel Stent is that the crosslinked gelatin material of the device often provokes inflammation and scarring, which can require surgical intervention to restore flow. The XEN Gel Stent is also known to, at times, be expelled or rejected from the eye through the conjunctiva; however, it rarely migrates into the anterior chamber.

FIG. 1 shows a schematic view of a PRESERFLO® MicroShunt device 1 secured under the conjunctiva/Tenon's capsule (not shown) and over the sclera of a human eye. The device 1 includes a tube 2 with opposed fins 3 located midway down the length of the tube 2. This device is best described in U.S. Pat. Nos. 7,431,709, 7,594,899, 7,837,644, 9,044,301, 9,101,444, 9,889,042, included in their entirety within. The tube 3 has an internal lumen that extends from a tapered or beveled distal end 4 to a proximal end 5 and provides a channel that drains aqueous humor from the anterior chamber of the eye to a drainage bleb.

The lumen of the tube 1 is designed to be sufficiently small to limit flow through the tube and prevent hypotony, and can range from 60 μm to 100 μm; preferably 70 μm. The outer diameter is set to approximately 350 μm to be sufficiently small not to erode tissue yet have sufficient columnar strength to be pushed with a forceps through a needle tract formed under the limbus. The fins 3 serve primarily as an obstruction or anchor to prevent the tube from migrating into the anterior chamber (it also serves as a cork to prevent peri-annular leakage).

FIG. 2 is a sagittal view of a human eye showing the PRESERFLO® MicroShunt device 1 implanted into the eye. The tube 2 rests under the conjunctiva and Tenon's capsule, and the fins 2 are lodged in a scleral pocket incised in the sclera. The internal lumen of tube 1 extends from the distal end 4 to the proximal end 5. The distal end 4 (lumen entrance) is deployed in the anterior chamber. The proximal end 5 (lumen exit) is deployed under the conjunctiva and Tenon's capsule where the accumulating reservoir forms a small blister called a bleb.

The PRESERFLO® MicroShunt device 1 is made entirely out of a bioinert flexible polymeric material called poly(styrene-block-isobutylene-block-styrene) (“SIBS”) which provokes clinically insignificant inflammation and tissue reaction. In short, it does not significantly encapsulate as does the XEN Gel Stent. And, in the absence of the fins 3, the device will migrate into the anterior chamber of the eye almost 100% of the time.

The PRESERFLO® MicroShunt device 1 is implanted using an ab externo procedure where the conjunctiva is dissected from the sclera and the distal end 4 of tube 1 is introduced through a needle tract (tissue passageway) formed in the sclera connecting to the anterior chamber of the eye. Because the PRESERFLO® MicroShunt device 1 has fins 3, it cannot be folded down and loaded into a needle and injected in the eye. The best that can be done is to first form a needle tract and then thread the PRESERFLO® MicroShunt device 1 through the needle tract with a forceps or with a “holder” as is described in US 2022/0133538 A1 with the fins 3 resting either outside the needle tract or in a pocket pre-formed with a knife to enable placement of the fins 3 and securing it to the sclera.

Forming the needle tract and threading the PRESERFLO® MicroShunt device 1 through the needle tract can be difficult, particularly for cases where collagen threads cross the needle tract or the needle tract collapses preventing the PRESERFLO® MicroShunt device from sliding through it. In addition, the PRESERFLO® MicroShunt device 1 cannot be implanted using an ab interno procedure as forming a needle tract as part of an ab interno procedure and then finding it to thread the PRESERFLO® MicroShunt device 1 therethrough is impractical, not to mention that the fins 3 would be on the wrong side of the needle tract and the device will migrate into the eye.

The suprachoroidal space is a space in the eye that lies between the sclera and the choroid. It is known that aqueous humor in the suprachoroidal space can drain therefrom and cause a reduction in intraocular pressure. Although it is not well understood where aqueous humor drains once it reaches the suprachoroidal space, there are references to aqueous humor draining into the choroid vessels as well as into the venous plexus of the sclera and to the episcleral veins.

Alcon Laboratories, Inc. of Fort Worth, Texas developed the CyPass® Microstent that includes a tubular body with an internal lumen that drains aqueous humor from the anterior chamber of the eye into the suprachoroidal space of the eye to lower intraocular pressure in the eye. The CyPass® Microstent was formed of a polyimide (PI) polymer. The tubular body had an inner diameter of 300 microns, outer diameter of 430 microns, and overall length of 6.35 mm. The device include three (3) exterior retention rings formed on the exterior surface of the tubular body to prevent migration of device after implantation and serve as landmarks during placement. The tubular body also had sixty-four (64) fenestrations or orifices through the tubular body into the internal lumen to ensure that the device would continue to drain aqueous humor in case that the distal end of lumen was blocked. The CyPass® Microstent was voluntarily withdrawn from the market in August 2018, based on analysis of completed data set at five years post-surgery which showed that, at five years, patients implanted with the CyPass Micro-Stent experienced statistically significant endothelial cell loss as compared to patients who underwent cataract surgery alone.

iSTAR Medical of Wavre, Belgium has developed the MINIject® device that has been approved in Europe for the treatment of glaucoma. The MINIject® device includes a lumen-less elongate body formed from a porous medical grade silicon material that drains aqueous humor from the anterior chamber of the eye into the suprachoroidal space of the eye to lower intraocular pressure in the eye. The elongate body of the MINIject® device does not have any features that prevent migration of device into the anterior chamber after implantation, and such migration can be problematic. For example, such migration of the device can inhibit drainage of aqueous humor and thus thwart treatment of the glaucoma.

### BRIEF DESCRIPTION OF THE DRAWINGS

PRIOR ART FIG. 1 is a schematic diagram of a PRESERFLO® MicroShunt device secured under the conjunctiva/Tenon's capsule (not shown) and over the sclera of a human eye.

PRIOR ART FIG. 2 shows a sagittal view of the eye with the PRESERFLO® MicroShunt device of FIG. 1 implanted into the eye.

FIGS. 3 to 8 are schematic diagrams of exemplary embodiments of glaucoma drainage devices according to the present disclosure.

FIG. 9 is a schematic diagram of the glaucoma drainage device of FIG. 6 held within the distal end of a rigid hollow needle inserter.

FIG. 10 is a schematic diagram illustrating deployment of the glaucoma drainage device of FIG. 6 from the distal end of a rigid hollow needle inserter.

FIG. 11 is a schematic diagram of the glaucoma drainage device of FIG. 6 as deployed in in a needle tract through ocular tissue.

FIG. 12 is a schematic diagram of the glaucoma drainage device similar to the embodiment of FIG. 6 held within the distal end of a rigid hollow needle inserter.

FIG. 13 is a schematic diagram illustrating deployment of the glaucoma drainage device from the distal end of the rigid hollow needle inserter of FIG. 12.

FIG. 14 is a cross-section of the glaucoma drainage device of FIGS. 12 and 13 as deployed in a needle tract through ocular tissue (such as, for example, the needle tract shown in FIG. 11).

FIG. 15 is a schematic diagram of the glaucoma drainage device similar to the embodiment of FIG. 6 held within the distal end of another rigid hollow needle inserter.

FIG. 16 is a schematic diagram illustrating deployment of the glaucoma drainage device from the distal end of the rigid hollow needle inserter of FIG. 15.

FIGS. 17 to 19 are schematic diagrams of exemplary embodiments of glaucoma drainage devices according to the present disclosure.

### DETAILED DESCRIPTION OF THE PREFERRED EMBODIMENTS

The present disclosure includes a simple glaucoma drainage device that can be contained within a hollow needle inserter and deployed from the hollow needle inserter into the eye in one shot, eliminating the need to pre-form a needle tract. Furthermore, the glaucoma drainage device can be implanted into the eye using an ab externo procedure. Alternatively, the drainage device can be implanted into the eye using an ab interno procedure. These features can be beneficial to the patient.

For the purposes of this disclosure, an ab externo procedure implants a glaucoma drainage device into the eye by inserting the device through a tissue passageway leading to the anterior chamber of the eye. An ab interno procedure implants a glaucoma drainage device into the eye through an incision in the cornea, and then from a position inside the anterior chamber through a tissue passageway leading to drainage space outside the anterior chamber. In either the ab externo procedure or the ab interno procedure, the glaucoma drainage device shunts aqueous humor from the anterior chamber into drainage space, such as drainage space under the conjunctiva and Tenon's capsule or in suprachoroidal space (i.e., space between the sclera and choroid).

Furthermore, the term “distal” generally refers to the direction away from the user/doctor that implants a glaucoma drainage device. Conversely, the term “proximal” generally refers to the direction toward the user/doctor that implants a glaucoma drainage device.

In the below descriptions, reference will be made to an anchor or wire. In embodiments, the anchor or wire can be made from a metal such as nickel-titanium alloy, cobalt-chromium-nickel alloy, MP35N, titanium, stainless steel, tantalum, and the like.

The device including the anchor or wire can be housed within the distal end of a rigid hollow needle inserter and deployed therefrom to implant the device into the eye. The implantation of the device into the eye can be carried out using an ab externo procedure where the distal end of the hollow needle inserter penetrates from space outside the eye, under the limbus, into the anterior chamber to form a needle tract (i.e., tissue passageway) through ocular tissue connecting to the anterior chamber of the eye for deployment of the device in the eye. Alternatively, the implantation of the device into the eye can be carried out using an ab interno procedure where the distal end of the hollow needle inserter is first located inside the anterior chamber of the eye via a corneal incision and then penetrates from the anterior chamber to space outside the anterior chamber (such as to a drainage space) to form a needle tract (i.e., tissue passageway) through ocular tissue leading from the anterior chamber of the eye for deployment of the device in the eye. When the device is loaded inside the distal end of the hollow needle inserter, the wire or anchor (or part thereof) is retained in a compressed state. With the distal end of the hollow needle inserter positioned in the needle tract with the anchor or wire disposed within the needle tract, the glaucoma drainage device can be deployed from the distal end of the hollow needle inserter. In this configuration, the wire or anchor (or part thereof) is configured to automatically recoil and expand outwardly relative to the compressed state (e.g., by self-expansion) and contact or snag ocular tissue of the needle tract to mechanically secure or fixate the device at the position of deployment in the eye, which can avoid unwanted migration of the device into the anterior chamber of the eye. In embodiments, the anchor or wire of the device can be formed for wire or coiled wire having a diameter ranging from 0.001 inches to 0.008 inches, and preferably between 0.002 inches and 0.004 inches. When the device is implanted into the eye using an ab externo procedure, the distal end of the drainage tube of the device is located in the anterior chamber of the eye, and the proximal end of the drainage tube is located in a drainage space, such as a drainage space under the conjunctiva and Tenon's capsule or in suprachoroidal space. When the device is implanted into the eye using an ab interno procedure, the distal end of the drainage tube of the device is located in a drainage space, such as a drainage space under the conjunctiva and Tenon's capsule or in suprachoroidal space, and the proximal end of the drainage tube is located inside the anterior chamber of the eye.

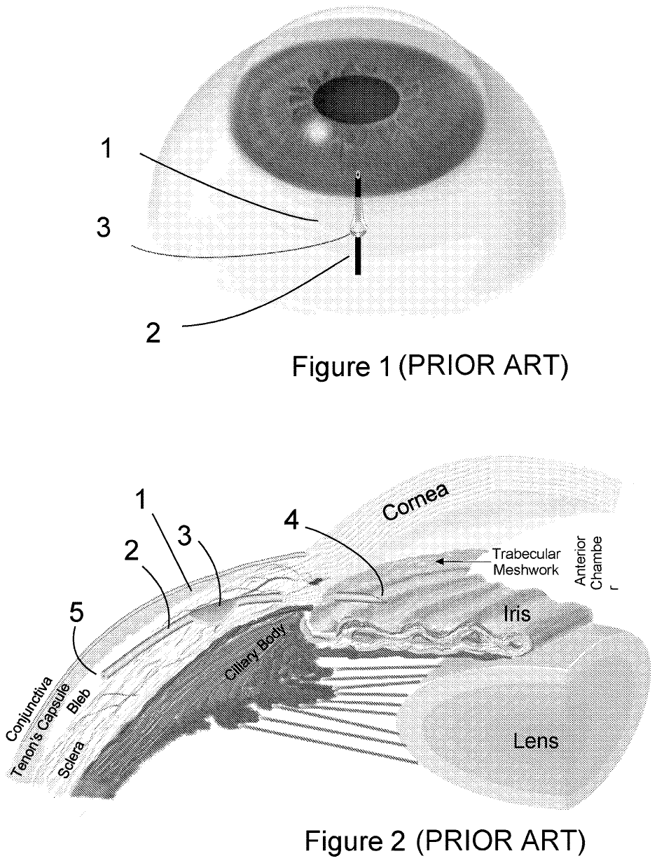

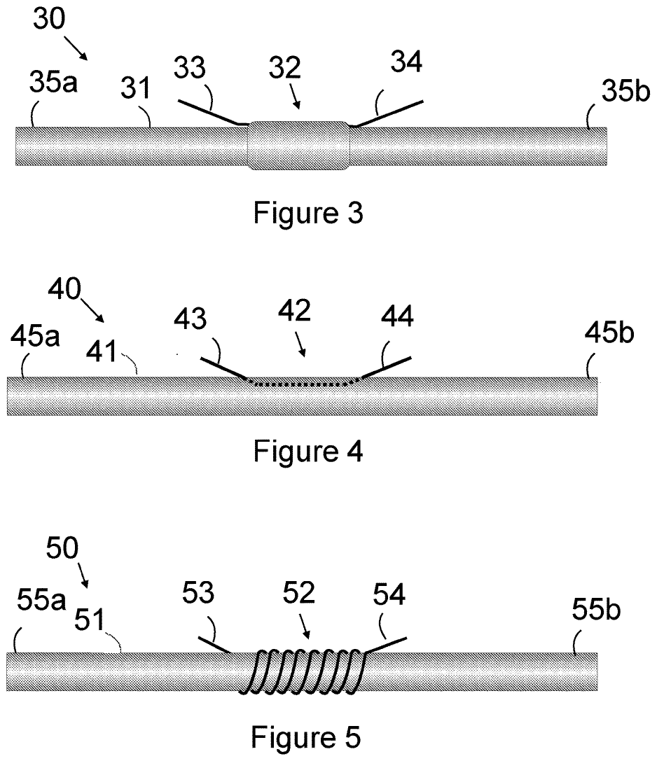

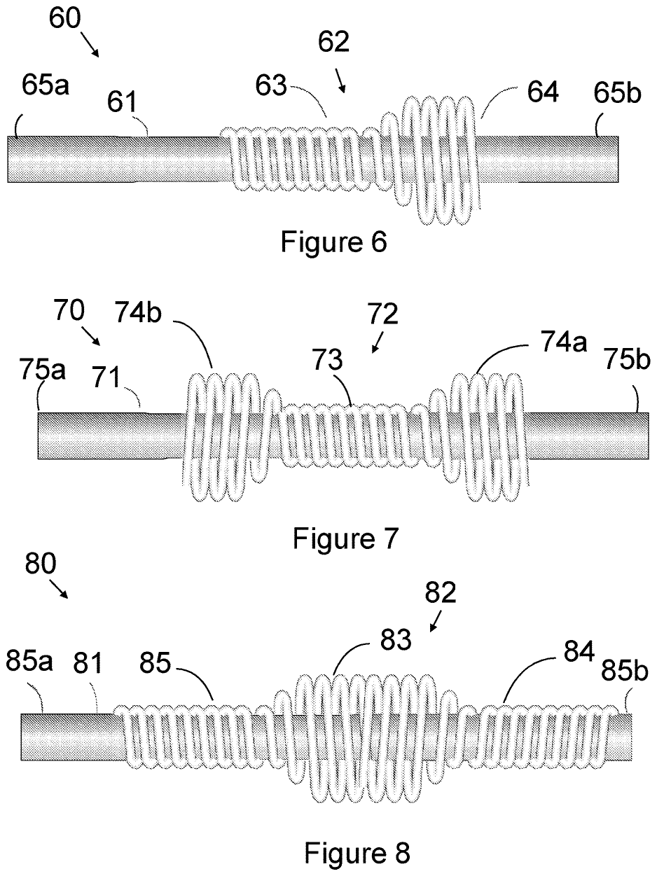

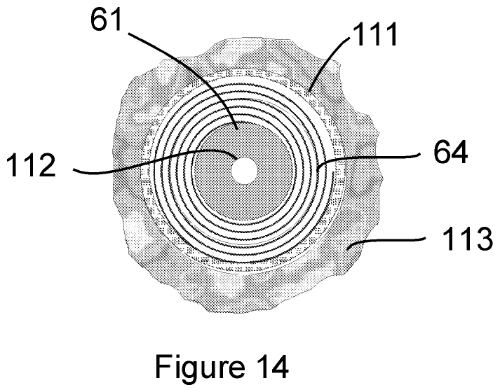

FIG. 3 shows an embodiment of an exemplary glaucoma drainage device 30 of the present disclosure having a drainage tube 31 that supports a thin sleeve 32 placed over the tube 31 between opposed ends 35a, 35b. The drainage tube 31 has an internal lumen (similar to lumen 112 of FIG. 14) that extends between the opposed ends 35a, 35b and provides a channel that drains aqueous humor from the anterior chamber of the eye to a drainage site or bleb. Sleeve 32 holds in place wire parts 33, 34 that function as an anchor or wire as described herein. The wire parts 33, 34 can be bent radially away from the longitudinal axis of tube 31 as shown. When device 30 is loaded inside the distal end of a hollow needle inserter, the wire parts 33, 34 can be retained in a compressed state. With the distal end of the hollow needle inserter positioned in the needle tract with the wire parts 33, 34 disposed within the needle tract, the glaucoma drainage device 30 can be deployed from the distal end of the hollow needle inserter. In this configuration, the wire parts 33, 34 can automatically recoil and expand radially outward relative to the compressed state (e.g., by self-expansion) and contact or snag ocular tissue of the needle tract to mechanically secure or fixate device 30 at the position of deployment in the eye, which can avoid unwanted migration of the device into the anterior chamber of the eye. In embodiments, the wire parts 33, 34 can be formed of wire having a wire diameter ranging from 0.001 inches to 0.008 inches, and preferably between 0.002 inches and 0.004 inches. The wire parts 33, 34 can be two separate wires, or one continuous wire. In an alternative embodiment, the device 30 can employ only one wire (i.e., 33 or 34) that extends from one side of sleeve 32. Device 30 (including the wire parts 33 and/or 34) can be housed within the distal end of the hollow needle inserter and deployed therefrom to implant the device into the eye (similar to FIGS. 9 and 10). The deployment can be carried out as part of an ab externo procedure where the distal end of the hollow needle inserter penetrates through ocular tissue into the anterior chamber for deployment of device 30 in the eye. When the device 30 is implanted into the eye using the ab externo procedure, the distal end (e.g., end 35b) of the drainage tube 31 of the device 30 is located in the anterior chamber of the eye, and the proximal end (e.g., end 35a) of the drainage tube 31 is located in a drainage space, such as a drainage space under the conjunctiva and Tenon's capsule or in suprachoroidal space. Alternatively, the deployment can be carried out as part of an ab interno procedure where the distal end of the hollow needle inserter is first located inside the anterior chamber of the eye via a corneal incision and then penetrates ocular tissue to drainage space, such as a drainage space under the conjunctiva and Tenon's capsule or in suprachoroidal space, for deployment of device 30 in the eye. When the device 30 is implanted into the eye using the ab interno procedure, the distal end (e.g., end 35b) of the drainage tube 31 of the device 30 is located in the drainage space, such as a drainage space under the conjunctiva and Tenon's capsule or in suprachoroidal space, and the proximal end (e.g., end 35a) of the drainage tube 31 is located inside the anterior chamber of the eye.

FIG. 4 shows an embodiment of another exemplary glaucoma drainage device 40 of the present disclosure having a drainage tube 41 having an internal lumen (similar to lumen 112 of FIG. 14) that extends between opposed ends 45a, 45b and provides a channel that drains aqueous humor from the anterior chamber of the eye to a drainage site or bleb. Instead of a sleeve 32 (as used the embodiment of FIG. 3), wire parts 43, 44 are fed into a secondary channel (shown as a dotted line) in tube 41 or simply pierce the material that forms the wall of the tube 41 such that wire parts 43, 44 are mechanically secured to the central portion of the tube 41. The wire parts 43, 44 function as an anchor or wire as described herein. The wire parts 43, 44 can be bent radially away from the longitudinal axis of tube 41 as shown. When device 40 is loaded inside the distal end of a hollow needle inserter, the wire parts 43, 44 can be retained in a compressed state. With the distal end of the hollow needle inserter positioned in the needle tract with the wire parts 43, 44 disposed within the needle tract, the glaucoma drainage device 40 can be deployed from the distal end of the hollow needle inserter. In this configuration, the wire parts 43, 44 can automatically recoil and expand radially outward relative to the compressed state (e.g., by self-expansion) and contact or snag ocular tissue of the needle tract to mechanically secure or fixate the device 40 at the position of deployment in the eye, which can avoid unwanted migration of the device into the anterior chamber of the eye. In embodiments, the wire parts 43, 44 can be formed from wire having a wire diameter ranging from 0.001 inches to 0.008 inches, and preferably between 0.002 inches and 0.004 inches. The wire parts 43, 44 can be two separate wires, or one continuous wire. In an alternative embodiment, device 40 can employ only one wire (i.e., 43 or 44) that extends from the tube 41. Device 40 (including the wire parts 43 and/or 44) can be housed within the distal end of the hollow needle inserter and deployed therefrom to implant the device into the eye (similar to FIGS. 9 and 10). The deployment can be carried out as part an ab externo procedure where the distal end of the hollow needle inserter penetrates ocular tissue into the anterior chamber for deployment of device 40 in the eye. When the device 40 is implanted into the eye using the ab externo procedure, the distal end (e.g., end 45b) of the drainage tube 41 of the device 40 is located in the anterior chamber of the eye, and the proximal end (e.g., end 45a) of the drainage tube 41 is located in a drainage space, such as a drainage space under the conjunctiva and Tenon's capsule or in suprachoroidal space. Alternatively, the deployment can be carried out as part an ab interno procedure where the distal end of the hollow needle inserter is first located inside the anterior chamber of the eye via a corneal incision and then penetrates ocular tissue into a drainage space, such as a drainage space under the conjunctiva and Tenon's capsule or in suprachoroidal space. When the device 40 is implanted into the eye using the ab interno procedure, the distal end (e.g., end 45b) of the drainage tube 41 of the device 40 is located in the drainage space, such as a drainage space under the conjunctiva and Tenon's capsule or in suprachoroidal space, and the proximal end (e.g., end 45a) of the drainage tube 41 is located inside the anterior chamber of the eye.

FIG. 5 shows an embodiment of yet another exemplary glaucoma drainage device 50 of the present disclosure having a drainage tube 51 having an internal lumen (similar to lumen 112 of FIG. 14) that extends between opposed ends 55a, 55b and provides a channel that drains aqueous humor from the anterior chamber of the eye to a drainage site or bleb. A wire spring 52 wraps around the central portion of the tube 51 between the opposed ends 55a, 55b such that wire spring 52 is mechanically secured to the central portion of the tube 51. The wire spring 52 and its ends 53, 54 function as an anchor or wire as described herein. The wire parts 53, 54 can be bent radially away from the longitudinal axis of the tube 51 as shown. When device 50 is loaded inside the distal end of a hollow needle inserter, the wire parts 53, 54 can be retained in a compressed state. With the distal end of the hollow needle inserter positioned in the needle tract with the wire parts 53, 54 disposed within the needle tract, the glaucoma drainage device 50 can be deployed from the distal end of the hollow needle inserter. In this configuration, the wire parts 53, 54 can automatically recoil and expand radially outward relative to the compressed state (e.g., by self-expansion) and contact or snag ocular tissue of the needle tract to mechanically secure or fixate the device 50 at the position of deployment in the eye, which can avoid unwanted migration of the device into the anterior chamber of the eye. In embodiments, the wire spring 52 (including the wire parts 53, 54) can employ wire have a diameter ranging from 0.001 inches to 0.008 inches, and preferably between 0.002 inches and 0.004 inches. In an alternative embodiment, device 50 can employ only one wire part (i.e., 53 or 54) that extends from the tube 51. Device 50 (including the wire parts 53 and/or 54) can be housed within the distal end of the hollow needle inserter and deployed therefrom to implant the device into the eye (similar to FIGS. 9 and 10). The deployment can be carried out as part of an ab externo procedure where the distal end of the hollow needle inserter penetrates ocular tissue into the anterior chamber for deployment of device 50 in the eye. When the device 50 is implanted into the eye using the ab externo procedure, the distal end (e.g., end 55b) of the drainage tube 51 of the device 50 is located in the anterior chamber of the eye, and the proximal end (e.g., end 55a) of the drainage tube 51 is located in a drainage space, such as a drainage space under the conjunctiva and Tenon's capsule or in suprachoroidal space. Alternatively, the deployment can be carried out as part of an ab interno procedure where the distal end of the hollow needle inserter is first located inside the anterior chamber of the eye via a corneal incision and then penetrates ocular tissue into a drainage space, such as a drainage space under the conjunctiva and Tenon's capsule or in suprachoroidal space. When the device 50 is implanted into the eye using the ab interno procedure, the distal end (e.g., end 55b) of the drainage tube 51 of the device 50 is located in the drainage space, such as drainage space under the conjunctiva and Tenon's capsule or in suprachoroidal space, and the proximal end (e.g., end 55a) of the drainage tube 51 is located inside the anterior chamber of the eye.

Note that the embodiments of FIGS. 3, 4 and 5 are symmetrical with the wire or anchor extending from the exterior annular surface of the tube on both sides of fixture holding it in place. Embodiments can have a wire(s) or anchor(s) on only one side to the fixture. If one side contains a wire or anchor, it is preferentially oriented such that the wire or anchor is on the anterior chamber side of the tube to prevent migration of the device into the anterior chamber of the eye.

FIG. 6 shows an embodiment of still another exemplary glaucoma drainage device 60 of the present disclosure having a drainage tube 61 having an internal lumen (similar to lumen 112 of FIG. 14) that extends between opposed ends 65a, 65b and provides a channel that drains aqueous humor from the anterior chamber of the eye to a drainage site or bleb. A spring anchor 62 formed by coiled wire is configured to wrap around the central portion of the tube 61 between the opposed ends 65a, 65b. The spring anchor 62 has a lesser-diameter coil section 63 and larger-diameter flared coil section 64. The lesser-diameter coil section 63 is mechanically secured to the central portion of tube 61. Spring anchor 62 functions as an anchor or wire as described herein. When device 60 is loaded inside the distal end of a hollow needle inserter, the flared coil section 64 can be retained in a compressed state. With the distal end of the hollow needle inserter positioned in the needle tract with the spring anchor 62 disposed within the needle tract, the glaucoma drainage device 60 can be deployed from the distal end of the hollow needle inserter. In this configuration, the flared coil section 64 can automatically recoil and expand radially outward relative to the compressed state (e.g., by self-expansion) and contact ocular tissue of the needle tract to mechanically secure or fixate the device 60 at the position of deployment in the eye, which can avoid unwanted migration of the device into the anterior chamber of the eye. In embodiments, the spring anchor 62 (including the lesser-diameter coil section 63 and flared coil section 64) can employ wire have a diameter ranging from 0.001 inches to 0.008 inches, and preferably between 0.002 inches and 0.004 inches. The device 60 (including the spring anchor 62) can be housed within the distal end of the hollow needle inserter and deployed therefrom to implant the device into the eye (see FIGS. 9 and 10). The deployment can be carried out as part of an ab externo procedure where the distal end of the hollow needle inserter penetrates ocular tissue into the anterior chamber for deployment of device 60 in the eye. When the device 60 is implanted into the eye using the ab externo procedure, the distal end (e.g., end 65b) of the drainage tube 61 of the device 60 is located in the anterior chamber of the eye, and the proximal end (e.g., end 65a) of the drainage tube 61 is located in a drainage space, such as a drainage space under the conjunctiva and Tenon's capsule or in suprachoroidal space. Alternatively, the deployment can be carried out as part of an ab interno procedure where the distal end of the hollow needle inserter is first located inside the anterior chamber of the eye via a corneal incision and then penetrates ocular tissue into a drainage space, such as a drainage space under the conjunctiva and Tenon's capsule or in suprachoroidal space. When the device 60 is implanted into the eye using the ab interno procedure, the distal end (e.g., end 65b) of the drainage tube 61 of the device 60 is located in the drainage space, such as drainage space under the conjunctiva and Tenon's capsule or in suprachoroidal space, and the proximal end (e.g., end 65a) of the drainage tube 61 is located inside the anterior chamber of the eye.

FIG. 7 shows another exemplary glaucoma drainage device 70 of the present disclosure, which is a symmetrical version similar to the embodiment of FIG. 6 and has a spring anchor 72 formed by coiled wire that is configured to wrap around the central portion of the tube 71 between its opposed ends 75a, 75b. The tube 71 has an internal lumen (similar to lumen 112 of FIG. 14) that extends between the opposed ends 75a, 75b and provides a channel that drains aqueous humor from the anterior chamber of the eye to a drainage site or bleb. The spring anchor 72 has larger-diameter flared coil sections 74a and 74b that are disposed on either side of a lesser-diameter coil section 73. The spring anchor 72 functions as an anchor or wire as described herein. When device 70 is loaded inside the distal end of a hollow needle inserter, the flared coil sections 74a, 74b can be retained in a compressed state. With the distal end of the hollow needle inserter positioned in the needle tract with the spring anchor 72 disposed within the needle tract, the glaucoma drainage device 70 can be deployed from the distal end of the hollow needle inserter. In this configuration, the flared coil sections 74a, 74b can automatically recoil and expand radially outward relative to the compressed state (e.g., by self-expansion) and contact ocular tissue of the needle tract to mechanically secure or fixate the device 70 at the position of deployment in the eye, which can avoid unwanted migration of the device into the anterior chamber of the eye. In embodiments, the spring anchor 72 (including the lesser-diameter coil section 73 and flared coil sections 74a, 74b) can employ wire having a diameter ranging from 0.001 inches to 0.008 inches, and preferably between 0.002 inches and 0.004 inches. Device 70 including the spring anchor 72 can be housed within the distal end of a hollow needle inserter and deployed therefrom to implant the device into the eye (similar to FIGS. 9 and 10). The deployment can be carried out as part of an ab externo procedure where the distal end of the hollow needle inserter penetrates ocular tissue into the anterior chamber for deployment of the device 70 in the eye. When the device 70 is implanted into the eye using the ab externo procedure, the distal end (e.g., end 75b) of the drainage tube 71 of the device 70 is located in the anterior chamber of the eye, and the proximal end (e.g., end 75a) of the drainage tube 71 is located in a drainage space, such as a drainage space under the conjunctiva and Tenon's capsule of the eye outside the eye. Alternatively, the deployment can be carried out as part of an ab interno procedure where the distal end of the hollow needle inserter is first located inside the anterior chamber of the eye via a cornel incision and then penetrates ocular tissue into a drainage space, such as a drainage space under the conjunctiva and Tenon's capsule of the eye, for deployment of the device 70 in the eye. When the device 70 is implanted into the eye using the ab interno procedure, the distal end (e.g., end 75b) of the drainage tube 71 of the device 70 is located in the drainage space, such as a drainage space under the conjunctiva and Tenon's capsule of the eye, and the proximal end (e.g., end 75a) of the drainage tube 71 is located inside the anterior chamber of the eye.

FIG. 8 shows yet another exemplary glaucoma drainage device 80 of the present disclosure, which is similar to the embodiment of FIGS. 6 and 7 and has a spring anchor 82 formed by coiled wire that is configured to wrap around the central portion of the tube 81 between its opposed ends 85a, 85b. The tube 81 has an internal lumen (similar to lumen 112 of FIG. 14) that extends between the opposed ends 85a, 85b and provides a channel that drains aqueous humor from the anterior chamber of the eye to a drainage site or bleb. The spring anchor 82 has lesser-diameter coil sections 84, 85 disposed on either side of flared coil section 83. The lesser-diameter coil sections 84, 85 and the flared coil section 83 function as an anchor or wire as described herein. When device 80 is loaded inside the distal end of a hollow needle inserter, the flared coil section 83 can be retained in a compressed state. With the distal end of the hollow needle inserter positioned in the needle tract with the spring anchor 82 disposed within the needle tract, the glaucoma drainage device 80 can be deployed from the distal end of the hollow needle inserter. In this configuration, the flared coil section 83 can automatically recoil and expand radially outward relative to the compressed state (e.g., by self-expansion) and contact ocular tissue of the needle tract to mechanically secure or fixate the device 80 at the position of deployment in the eye, which can avoid unwanted migration of the device into the anterior chamber of the eye. In embodiments, the lesser-diameter coil sections 84, 85 and the flared coil section 83 can employ wire having a diameter ranging from 0.001 inches to 0.008 inches, and preferably between 0.002 inches and 0.004 inches.

The device 80 can be housed within the distal end of the hollow needle inserter and deployed therefrom to implant the device into the eye (similar to FIGS. 9 and 10). The deployment can be carried out as part of an ab externo procedure where the distal end of the hollow needle inserter penetrates ocular tissue into the anterior chamber for deployment of device 80 in the eye. When the device 80 is implanted into the eye using the ab externo procedure, the distal end (e.g., end 85b) of the drainage tube 81 of the device 80 is located in the anterior chamber of the eye, and the proximal end (e.g., end 85a) of the drainage tube 81 is located in a drainage space, such as a drainage space under the conjunctiva and Tenon's capsule or in suprachoroidal space. Alternatively, the deployment can be carried as part of an ab interno procedure where the distal end of the hollow needle inserter is first located inside the anterior chamber of the eye via a cornel incision and then penetrates ocular tissue into a drainage space, such as a drainage space under the conjunctiva and Tenon's capsule or in suprachoroidal space. When the device 80 is implanted into the eye using the ab interno procedure, the distal end (e.g., end 85b) of the drainage tube 81 of the device 80 is located in the drainage space, such as drainage space under the conjunctiva and Tenon's capsule or in suprachoroidal space, and the proximal end (e.g., end 85a) of the drainage tube 81 is located inside the anterior chamber of the eye.

FIG. 9 shows the distal end of an exemplary rigid hollow needle inserter 90 with a sharp tissue-piercing needle tip 91. The distal end of the rigid hollow needler inserter 90 defines a chamber that houses the device 60 or part thereof (embodiment of FIG. 6), which includes the spring anchor 62 (including the lesser-diameter coil section 63 and the flared coil section 64 in a compressed state). The rigid hollow needle inserter 90 with the sharp tissue-piercing needle tip 91 is used to pierce through ocular tissue to form a needle tract that will receive the drainage device 60 and spring anchor 62 during implantation into the eye. The hollow needle inserter 90 can include a pusher rod 96 disposed adjacent the proximal end 65a of the tube 61 of the device 60. The distal end of the pusher rod 96 can be configured to push on the proximal end 65a to deploy the device 60 from the distal chamber of the hollow needle inserter 90 as shown in FIG. 10. The pusher rod 96 can be configured to support an optional stiffener stylus 97 that extends from the distal end of the pusher rod 96 into and through the lumen of the device 60 as shown. The stiffener stylus 97 can be formed from a metal filament having a diameter less than the diameter of the lumen of the device 60. The stylus stiffener stylus 97 can support the tube 61 of device 60 during deployment to minimize buckling of the tube 61 during such deployment which can occur due to flexible construction of the tube 61.

FIG. 10 shows the rigid hollow needle inserter 90 of FIG. 9 where the pusher rod 96 is held stationery and the needle tip 91 is slid back relative to the pusher rod 96 to release the flared section 64 of the anchor 62 as well as the rest of drainage device 60 for deployment of the device in the eye. When the device is deployed from the distal chamber of the rigid hollow needle inserter 90 into the eye, the flared coil section 64 can be configured to automatically recoil and expand radially outward relative to the compressed state (e.g., by self-expansion) and contact ocular tissue of the needle tract formed by the hollow needle inserter 90 to mechanically secure or fixate the device 60 at the position of deployment in the eye, which can avoid unwanted migration of the device into the anterior chamber of the eye. The deployment can be carried out as part of an ab externo procedure where the needle tip 91 of the rigid needle inserter 90 penetrates into ocular tissue into the anterior chamber for deployment of the device 60 in the eye. Alternatively, the deployment can be carried out as part of an ab interno procedure where the needle tip 91 of the needle inserter 90 is first located inside the anterior chamber via a cornel incision and then penetrates ocular tissue into a drainage space, such as a drainage space under the conjunctiva and Tenon's capsule or in suprachoroidal space.

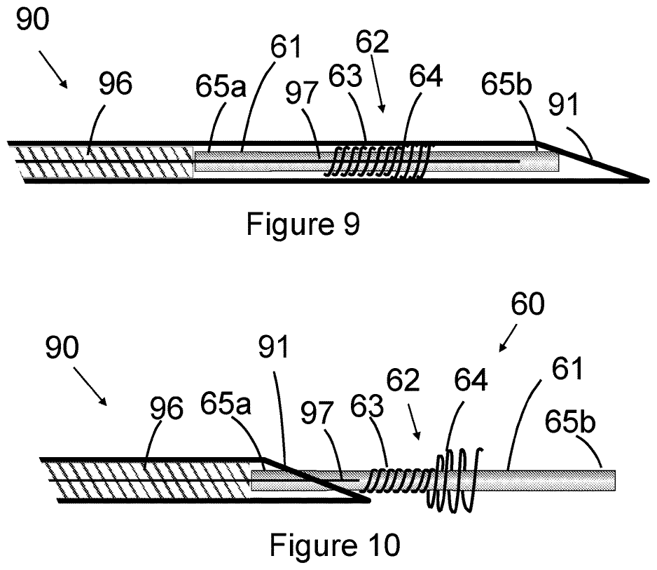

FIG. 11 shows a sagittal image of the eye with drainage device 60 secured by the flared coil section 64 of anchor 62 within the needle tract formed by the hollow needle inserter of FIG. 10.

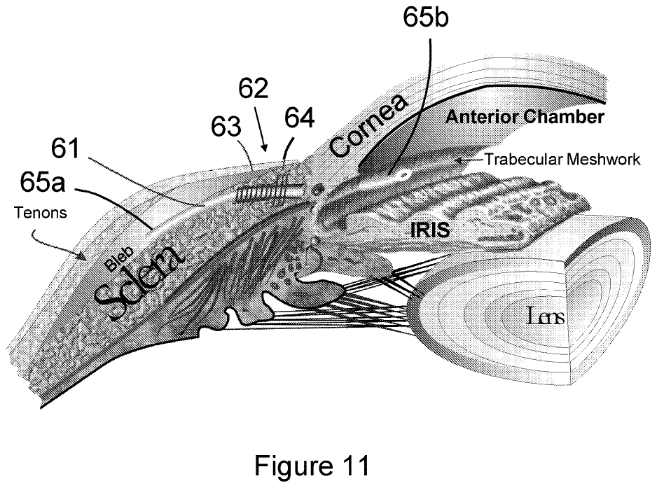

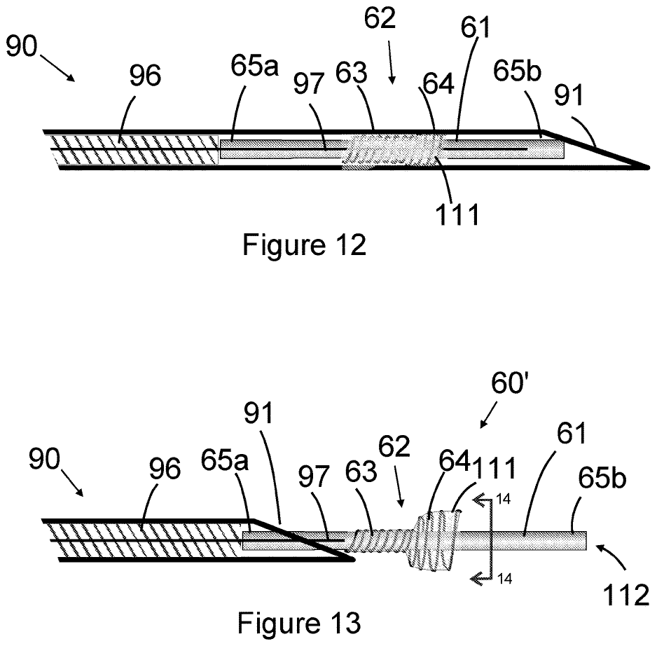

FIGS. 12 and 13 show yet another exemplary glaucoma drainage device 60′ of the present disclosure, which is similar to the embodiment of FIG. 6. In this embodiment, the coil sections 63, 64 of the spring anchor 62 are coated or lined with a thin sheath 111. One part of the sheath 111 can be glued or bonded to the outer surface of the coil section 63 and/or tube 61 to form a seal around the entire circumference of the tube 61, with another part of the sheath 111 configured to expand with the flared coil section 64 about the entire circumferential perimeter of the tube 61. In the expanded state of the flared coil section 64, the sheath 111 can interface to surrounding tissue such that sheath 111 obstructs peri-annular flow or leakage of aqueous humor. The sheath 111 can be made from a porous or non-porous flexible elastomeric polymeric fabric, such as a fabric formed from expanded polytetrafluoroethylene (xPTFE), Dacron®, polyurethane, SIBS, and the like. The sheath 111 can be similar to the sheaths employed in stent-grafts or endoluminal-grafts, In this embodiment, the spring anchor 62 can serve as a mechanism to support an umbrella-like sheath 111 that when opened in the needle tract prevents peri-annular flow or leakage of aqueous humor.

FIG. 12 shows the distal end of an exemplary rigid hollow needle inserter 90 with a sharp tissue-piercing needle tip 91. The distal end of the rigid hollow needler inserter 90 defines a chamber that houses the device 60′, which includes the spring anchor 62 with sheath 111. The rigid hollow needle inserter 90 with the sharp tissue-piercing needle tip 91 is used to pierce through ocular tissue to form a needle tract that will receive the drainage device 60′ and spring anchor 62 and sheath 111 during implantation into the eye. The hollow needle inserter 90 can include a pusher rod 96 disposed adjacent the proximal end 65a of the tube 61 of the device 60′. The distal end of the pusher rod 96 can be configured to push on the proximal end 65a to deploy the device 60′ from the distal chamber of the hollow needle inserter 90 as shown in FIG. 13. The pusher rod 96 can be configured to support an optional stiffener stylus 97 that extends from the distal end of the pusher rod 96 into and through the lumen of the device 60 as shown. The stiffener stylus 97 can be formed from a metal filament having a diameter less than the diameter of the lumen of the device 60′. The stylus stiffener stylus 97 can support the tube 61 of device 60′ during deployment to minimize buckling of the tube 61 during such deployment which can occur due to flexible construction of the tube 61.

FIG. 13 shows the rigid hollow needle inserter 90 of FIG. 12 where the pusher rod 96 is held stationery and the needle tip 91 is slid back relative to the pusher rod 96 to release the flared section 64 of the anchor 62 and the sheath 111 as well as the rest of drainage device 60′ for deployment of the device in the eye. When the device 60′ is deployed from the distal end of the rigid hollow needle inserter 90 into the eye, the flared coil section 64 can be configured to automatically recoil and expand radially outward relative to the compressed state (e.g., by self-expansion) and contact ocular tissue of the needle tract to mechanically secure or fixate the device at the position of deployment in the eye, which can avoid unwanted migration of the device into the anterior chamber of the eye. In this configuration, the spring anchor 62 can serve as a mechanism to support the umbrella-like sheath 111 that when opened in the needle tract prevents unwanted peri-annular leakage of aqueous humor around the exterior annular surface of the tube 61 of the device 60′, which could otherwise flow out of the anterior chamber of the eye in an uncontrolled manner. The deployment can be carried out as part of an ab externo procedure where the needle tip 91 of the hollow needle inserter 90 penetrates ocular tissue into the anterior chamber for deployment of the device in the eye. When the device 60′ is implanted into the eye using the ab externo procedure, the distal end (e.g., end 65b) of the drainage tube 61 of the device 60′ is located in the anterior chamber of the eye, and the proximal end (e.g., end 65a) of the drainage tube 61 is located in a drainage space, such as a drainage space under the conjunctiva and Tenon's capsule or in suprachoroidal space. Alternatively, the deployment can be carried as part of an ab interno procedure where the distal end of the hollow needle inserter 90 is first located inside the anterior chamber via a corneal incision and then penetrates ocular tissue into a drainage space, such as a drainage space under the conjunctiva and Tenon's capsule or in suprachoroidal space. When the device 60′ is implanted into the eye using the ab interno procedure, the distal end (e.g., end 65b) of the drainage tube 61 of the device 60′ is located in the drainage space, such as drainage space under the conjunctiva and Tenon's capsule or in suprachoroidal space, and the proximal end (e.g., end 65a) of the drainage tube 61 is located inside the anterior chamber of the eye.

FIG. 14 is a cross-section at 14-14 of FIG. 13, showing the device 60′ in the needle tract through ocular tissue 113. At the center is the tube 61 with lumen 112, surrounded by the spring anchor 62. The thin sheath 111 is disposed about the outer surface of the flared section 64 of the anchor 62. The sheath 111 can extend from the exterior surface of the tube 61 and surround the circumferential perimeter of the tube 61 to interface to the surrounding tissue 113, which effectively prevents an unwanted flow of aqueous humor in the needle tract outside of tube 92 and within the needle tract formed in the tissue 113. This unwanted flow of aqueous humor is commonly referred to as peri-annular leakage of aqueous humor.

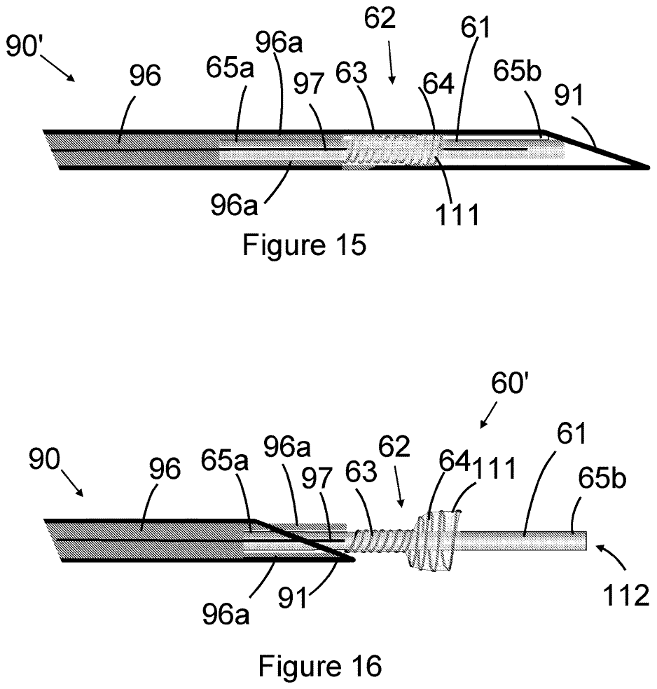

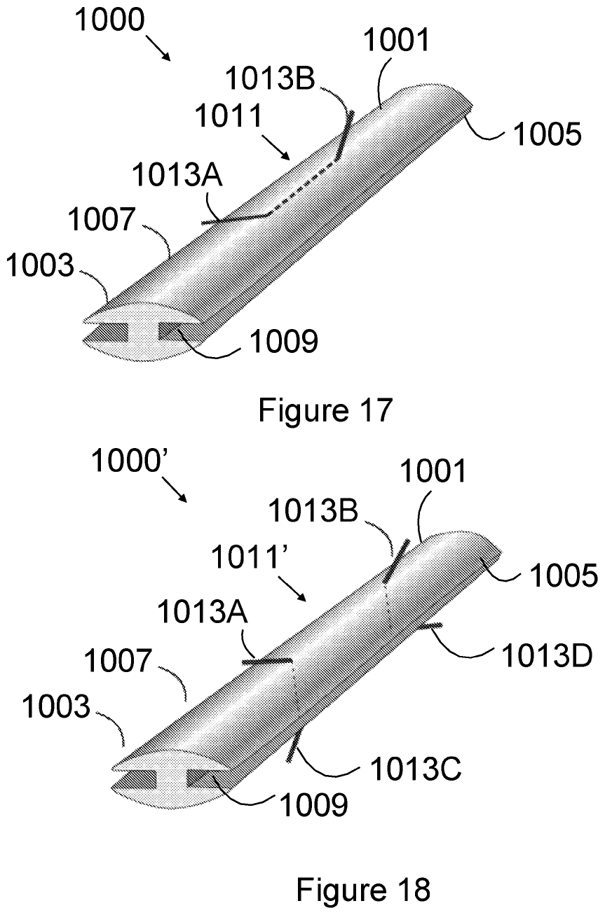

FIGS. 15 and 16 show an exemplary rigid hollow needle inserter 90′ used for deployment of the glaucoma drainage device 60′ of FIGS. 12 and 13. FIG. 15 shows the distal end of the rigid hollow needle inserter 90′ with a sharp tissue-piercing needle tip 91. The distal end of the rigid hollow needler inserter 90′ defines a chamber that houses the device 60′, which includes the spring anchor 62 with sheath 111. The rigid hollow needle inserter 90′ with the sharp tissue-piercing needle tip 91 is used to pierce through ocular tissue to form a needle tract that will receive the drainage device 60′ and spring anchor 62 and sheath 111 during implantation into the eye. The hollow needle inserter 90′ can include a pusher rod 96′ with one or more projections or other features 96a disposed at the distal end of the pusher rod 96. The projections or other features 96a are disposed about and/or surround the proximal end 65a of the tube 61 of the device 60′. The projections or other features 96a can be configured to push on the proximal end (or other part) of the spring anchor 62 to deploy the device 60′ from the distal chamber of the hollow needle inserter 90′ as shown in FIG. 16. The pusher rod 96 can be configured to support an optional stiffener stylus 97 that extends from the distal end of the pusher rod 96 into and through the lumen of the device 60 as shown. The stiffener stylus 97 can be formed from a metal filament having a diameter less than the diameter of the lumen of the device 60′. The stylus stiffener stylus 97 can support the tube 61 of device 60′ during deployment to minimize buckling of the tube 61 during such deployment which can occur due to flexible construction of the tube 61.

FIG. 16 shows the rigid hollow needle inserter 90′ of FIG. 15 where the pusher rod 96′ is held stationery and the needle tip 91 is slid back relative to the pusher rod 96′ to release the flared section 64 of the anchor 62 and the sheath 111 as well as the rest of drainage device 60′ for deployment of the device in the eye. When the device 60′ is deployed from the distal end of the rigid hollow needle inserter 90′ into the eye, the flared coil section 64 can be configured to automatically recoil and expand radially outward relative to the compressed state (e.g., by self-expansion) and contact ocular tissue of the needle tract to mechanically secure or fixate the device at the position of deployment in the eye, which can avoid unwanted migration of the device into the anterior chamber of the eye. In this configuration, the spring anchor 62 can serve as a mechanism to support the umbrella-like sheath 111 that when opened in the needle tract prevents unwanted peri-annular leakage of aqueous humor around the exterior annular surface of the tube 61 of the device 60′, which could otherwise flow out of the anterior chamber of the eye in an uncontrolled manner. The deployment can be carried out as part of an ab externo procedure where the needle tip 91 of the hollow needle inserter 90′ penetrates ocular tissue into the anterior chamber for deployment of the device in the eye. When the device 60′ is implanted into the eye using the ab externo procedure, the distal end (e.g., end 65b) of the drainage tube 61 of the device 60′ is located in the anterior chamber of the eye, and the proximal end (e.g., end 65a) of the drainage tube 61 is located in a drainage space, such as a drainage space under the conjunctiva and Tenon's capsule or in suprachoroidal space. Alternatively, the deployment can be carried as part of an ab interno procedure where the distal end of the hollow needle inserter 90′ is first located inside the anterior chamber via a corneal incision and then penetrates ocular tissue into a drainage space, such as a drainage space under the conjunctiva and Tenon's capsule or in suprachoroidal space. When the device 60′ is implanted into the eye using the ab interno procedure, the distal end (e.g., end 65b) of the drainage tube 61 of the device 60′ is located in the drainage space, such as drainage space under the conjunctiva and Tenon's capsule or in suprachoroidal space, and the proximal end (e.g., end 65a) of the drainage tube 61 is located inside the anterior chamber of the eye.

In other embodiments, the spring anchors 72, 82 of the devices of FIGS. 7 and 8 can be equipped with a thin sheath similar to sheath 111 of FIG. 12. In this configuration, one or more parts of the sheath can be glued or bonded to the outer surface of the tube of the device to form a seal around the entire circumference of the tube, with another part(s) of the sheath configured to expand with the flared coil section(s) of the device about the entire circumferential perimeter of the tube. In the expanded state of the flared coil section(s), the sheath can interface to surrounding tissue such that sheath obstructs peri-annular flow or leakage of aqueous humor. The sheath can be made from a porous or non-porous flexible elastomeric polymeric fabric, such as fabric made from expanded polytetrafluoroethylene (xPTFE), Dacron®, polyurethane, SIBS, and the like. The sheath can be similar to the sheaths employed in stent-grafts or endoluminal-grafts, In these embodiments, the spring anchor can serve as a mechanism to support the expandable sheath that when opened in the needle tract prevents peri-annular flow or leakage of aqueous humor.

The hollow needle inserter as described herein can be used to deploy the glaucoma drainage devices of FIGS. 3, 4, 5, 7 and 8 in a manner similar to that described above with respect to FIGS. 9, 10, 12 and 13.

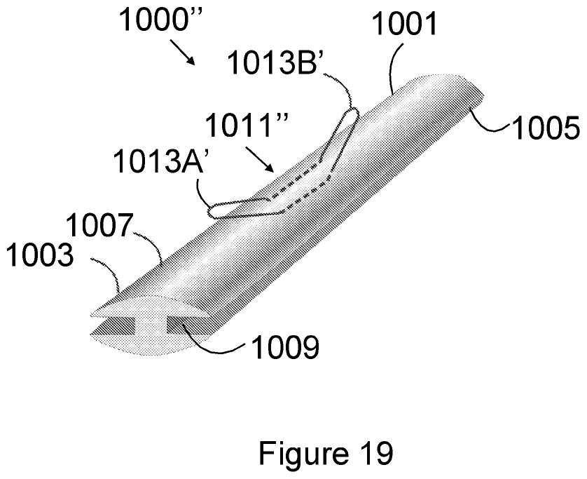

FIG. 17 shows an embodiment of a lumen-less glaucoma drainage device 1000 that is configured for implantation into suprachoroidal space of the eye. In the embodiment shown, the device 1000 is comprised of a lumen-less elongate body 1001 with opposed ends 1003, 1005. The lumen-less body 1001 is configured to enable aqueous humor to diffuse from the anterior chamber into the suprachoroidal space of the eye.

In the embodiment shown, the lumen-less body 1001 has one or more outer surfaces 1007 that define at least one open groove or channel 1009 that extend parallel to the longitudinal axis A-A of the elongate body 1001 between a point at or near end 1003 and a point at or near end 1005. The groove or channel 1009 enables aqueous humor to flow therein alongside the outer surface(s) of the body 1001 and diffuse from the anterior chamber into the suprachoroidal space of the eye. In alternate embodiments (not shown), the lumen-less elongate body 1001 can be formed from a porous material that permits flow of aqueous humor into and through the elongate body to drain aqueous humor from the anterior chamber of the eye into the suprachoroidal space.

The device 1000 further includes an anchor 1011 formed by wire parts 1013A, 1013B, which function as an anchor or wire similar to the embodiment of FIG. 3 as described herein. The wire parts 1013A, 1013B are disposed on one side of the body 1001 in the central portion of the body 1001 between the opposed ends 1003, 1005. The wire parts 1013A, 1013B can be bent radially away from the longitudinal axis of the body 1001 and toward the opposed ends 1003, 1005 as shown. When device 1000 is loaded inside the distal end of a hollow needle inserter, the wire parts 1013A, 1013B can be retained in a compressed state. With the distal end of the hollow needle inserter positioned in the needle tract with the wire parts 1013A, 1013B disposed within the needle tract, the glaucoma drainage device 1000 can be deployed from the distal end of the hollow needle inserter. In this configuration, the wire parts 1013A, 1013B can automatically recoil and expand radially outward relative to the compressed state (e.g., by self-expansion) and contact or snag ocular tissue of the needle tract to mechanically secure or fixate device 1000 at the position of deployment in the eye, which can avoid unwanted migration of the device into the anterior chamber of the eye. In embodiments, the wire parts 1013A, 1013B can be formed of wire having a wire diameter ranging from 0.001 inches to 0.008 inches, and preferably between 0.002 inches and 0.004 inches. The wire parts 1013A, 1013B can be two separate wires, or one continuous wire. In an alternative embodiment, the device 1000 can employ only one wire (i.e., 1013A or 1013B) that extends from the body 1001. Device 1000 (including the wire parts 1013A and/or 1013B) can be housed within the distal end of the hollow needle inserter and deployed therefrom to implant the device into the eye (similar to FIGS. 9 and 10). The deployment can be carried out as part of an ab interno procedure where the distal end of the hollow needle inserter is first located inside the anterior chamber of the eye via a corneal incision and then penetrates ocular tissue to suprachoroidal space (drainage space), for deployment of device 1000 in the eye. When the device 1000 is implanted into the eye using the ab interno procedure, the distal end (e.g., end 1005) of the body 1001 is located in the suprachoroidal space, i.e., drainage space, and the proximal end (e.g., end 1003) of the body 1001 is located inside the anterior chamber of the eye. Alternatively, the deployment can be carried out as part of an ab externo procedure where the distal end of the hollow needle inserter penetrates ocular tissue into the anterior chamber for deployment of device 1000 in the eye. When the device 1000 is implanted into the eye using the ab externo procedure, the distal end (e.g., end 1005) of the body 1001 is located in the anterior chamber of the eye, and the proximal end (e.g., end 1003) of the body 1001 is located in suprachoroidal space, i.e., drainage space. Alternatively, the deployment can be carried as part of an ab interno procedure where the distal end of the hollow needle inserter is first located inside the anterior chamber of the eye via a cornel incision and then penetrates ocular tissue into suprachoroidal space, i.e., drainage space.

FIG. 18 shows another embodiment of a lumen-less glaucoma drainage device 1000′ that is configured for implantation into suprachoroidal space of the eye.

The body of device 1000′ is identical to the body 1001 of device 1000 of FIG. 17. The device 1000′ further includes an anchor 1011′ formed by wire parts 1013A, 1013B, 1013C, 1013D which function as an anchor or wire similar to the embodiment of FIG. 3 as described herein. The wire parts 1013A, 1013B are disposed on one side of the body 1001 in the central portion of the body 1001 between the opposed ends 1003, 1005. The wire parts 1013A, 1013B can be bent radially away from the longitudinal axis of the body 1001 and toward the opposed ends 1003, 1005 as shown. The wire parts 1013C, 1013D are disposed on the opposite side of the body 1001 (relative to the wire parts 1013A, 1013B) in the central portion of the body 1001 between the opposed ends 1003, 1005.

The wire parts 1013C, 1013D can be bent radially away from the longitudinal axis of the body 1001 and toward the opposed ends 1003, 1005 as shown.

When device 1000′ is loaded inside the distal end of a hollow needle inserter, the wire parts 1013A, 1013B, 1013C, 19013D can be retained in a compressed state. With the distal end of the hollow needle inserter positioned in the needle tract with the wire parts 1013A, 1013B, 1013C, 1013D disposed within the needle tract, the glaucoma drainage device 1000′ can be deployed from the distal end of the hollow needle inserter. In this configuration, the wire parts 1013A, 1013B, 1013C, 1013D can automatically recoil and expand radially outward relative to the compressed state (e.g., by self-expansion) and contact or snag ocular tissue of the needle tract to mechanically secure or fixate device 1000′ at the position of deployment in the eye, which can avoid unwanted migration of the device into the anterior chamber of the eye. In embodiments, the wire parts 1013A, 1013B, 1013C, 1013D can be formed of wire having a wire diameter ranging from 0.001 inches to 0.008 inches, and preferably between 0.002 inches and 0.004 inches. The wire parts 1013A, 1013B can be two separate wires, or one continuous wire. Similarly, the wire parts 1013C, 1013D can be two separate wires, or one continuous wire. Device 1000′ (including the wire parts 1013A, 1013B, 1013C, 1013D) can be housed within the distal end of the hollow needle inserter and deployed therefrom to implant the device into the eye similar to the embodiment of FIG. 17 as described above.

FIG. 19 shows yet another embodiment of a lumen-less glaucoma drainage device 1000″ that is configured for implantation into suprachoroidal space of the eye. The body of device 1000″ is identical to the body 1001 of device 1000 of FIGS. 17 and 18. The device 1000″ further includes an anchor 1011″ formed by wire parts 1013A′, 1013B′, which function as an anchor or wire similar to the embodiment of FIG. 3 as described herein. The wire parts 1013A′, 1013B′ are disposed on one side of the body 1001 in the central portion of the body 1001 between the opposed ends 1003, 1005. The wire parts 1013A′, 1013B′ can be formed by wire half-loops that are bent radially away from the longitudinal axis of the body 1001 and toward the opposed ends 1003, 1005 as shown.

When device 1000″ is loaded inside the distal end of a hollow needle inserter, the wire parts 1013A′, 1013B′ can be retained in a compressed state. With the distal end of the hollow needle inserter positioned in the needle tract with the wire parts 1013A′, 1013B′ disposed within the needle tract, the glaucoma drainage device 1000″ can be deployed from the distal end of the hollow needle inserter. In this configuration, the wire parts 1013A′, 1013B′ can automatically recoil and expand radially outward relative to the compressed state (e.g., by self-expansion) and the ends of the half-loops of the wire parts 1013A′, 1013B′ can contact ocular tissue of the needle tract to mechanically secure or fixate device 1000′ at the position of deployment in the eye, which can avoid unwanted migration of the device into the anterior chamber of the eye. The ends of the half-loops of the wire parts 1013A′, 1013B′ can be designed to minimize or lessen damage to ocular tissue when deployed in the needle tract during implantation (as compared to the pointed ends of the wire parts of the anchors of FIGS. 17 and 18). In embodiments, the wire parts 1013A′, 1013B′ can be formed of wire having a wire diameter ranging from 0.001 inches to 0.008 inches, and preferably between 0.002 inches and 0.004 inches. The wire parts 1013A', 1013B′ can be two separate wires, or one continuous wire. Device 1000″ (including the wire parts 1013A′, 1013B′) can be housed within the distal end of the hollow needle inserter and deployed therefrom to implant the device into the eye similar to the embodiment of FIG. 17 as described above.

In embodiments, the body 1001 can have a shallow-curved I-beam-shaped cross-section as shown in FIGS. 17 to 19, which defines two grooves 1009 that extend parallel to one another on opposite sides of the body 1001 and parallel to the longitudinal axis of the body 1001. Alternatively, other cross-sectional shapes (such as a flat I-beam-shaped cross-section) or other suitable shapes can be used for the body 1001.

In other embodiments, any one of the anchor(s) or wires as shown in FIGS. 4 to 8 and described herein can be used in in place of (or in combination with) the anchor 1011 or 1011′ or 1011″ in the embodiments of FIGS. 17 to 19, respectively. Other suitable anchor or wire designs can be used as well.

The glaucoma drainage device(s) as described herein can be made from a bioinert flexible polymeric material. Exemplary flexible polymeric materials include polyurethane, polyisobutylene, polyisobutylene-urethane, poly(styrene-block-isobutylene-block-styrene) (“SIBS”), silicone rubber, PTFE, polyester, polysulfone, polyimide and any other material deemed bioinert in the body. The preferred material is SIBS. The lumen diameter of the device(s) can range from 40 μm to 100 μm; preferably 60 μm to 80 μm. The outer diameter of the device(s) can be 120 μm to 500 μm preferably 150 μm to 300 μm. The length of the device(s) can be 4 mm to 25 mm; preferably 5 mm to 9 mm. The needle gauge of the rigid hollow needle inserter used to deploy the device(s) can be 20 to 30 gauge, preferably 23 to 27 gauge. The device(s) can be placed using an ab interno procedure or an ab externo procedure to shunt aqueous humor from the anterior chamber to a drainage space, such as bleb formed under the conjunctiva and Tenon's capsule or in suprachoroidal space.

There have been described and illustrated herein several embodiments of a glaucoma drainage device and related systems and methods of treating glaucoma. Moreover, while particular configurations have been disclosed in reference to a glaucoma drainage device, it will be appreciated that other configurations could be used as well. It will therefore be appreciated by those skilled in the art that yet other modifications could be made to the provided invention without deviating from its spirit and scope as claimed.

1. A glaucoma drainage device comprising:
an elongate body configured to drain aqueous humor from the anterior chamber of the eye; and
at least one expandable anchor or wire that extends radially outward beyond the body and configured to fixate the body in the eye.

2. A glaucoma drainage device according to claim 1, wherein:
the at least one expandable anchor or wire is configured to automatically self-expand radially away from the body to fixate the body in the eye.

3. A glaucoma drainage device according to claim 1, wherein:
the at least one expandable anchor or wire comprises at least one wire part that extends radially outward beyond the body.

4. A glaucoma drainage device according to claim 3, wherein:
the at least one wire part has a wire diameter ranging from 0.001 inches to 0.008 inches.

5. A glaucoma drainage device according to claim 2, wherein:
the at least one expandable anchor or wire comprises at least one lesser-diameter coil section and at least one larger-diameter coil section, wherein the at least one lesser-diameter coil section wraps around the elongate body and is mechanically secured to the elongate body, and wherein the at least one larger-diameter coil section is configured to automatically self-expand radially away from body to fixate the body in the eye.

6. A glaucoma drainage device according to claim 5, wherein:
the at least one lesser-diameter coil section and the at least one larger-diameter coil section are formed by coiled wire having a wire diameter ranging from 0.001 inches to 0.008 inches.

7. A glaucoma drainage device according to claim 1, wherein:
the at least one expandable anchor or wire further comprises a sheath surrounding part of the expandable anchor or wire, wherein the sheath is configured to expand radially with the part of the expandable anchor or wire.

8. A glaucoma drainage device according to claim 7, wherein:
radial expansion of the sheath is configured to block peri-annular flow of aqueous humor around the body.

9. A glaucoma drainage device according to claim 7, wherein:
the sheath is made from a flexible elastomeric polymer.

10. A glaucoma drainage device according to claim 1, wherein:
the at least one expandable anchor or wire is made from a metal.

11. A glaucoma drainage device according to claim 10, wherein:
the metal is selected from the group including nickel-titanium alloy, cobalt-chromium-nickel alloy, MP35N, titanium, stainless steel, tantalum, and the like.

12. A glaucoma drainage device according to claim 1, wherein:
the elongate body is formed from a flexible polymeric material.

13. A glaucoma drainage device according to claim 1, wherein:
the at least one expandable anchor or wire is configured to fixate the body in a needle tract that connects to the anterior chamber of the eye.

14. A glaucoma drainage device according to claim 1, wherein:
the elongate body has a lumen that extends through the body.

15. A glaucoma drainage device according to claim 1, wherein:
the elongate body is lumen-less and has at least one flow channel defined by outer surfaces of the body or a porous material of the body.

16. A glaucoma drainage system including:
the glaucoma drainage device according to claim 1; and
a rigid needle inserter having a distal end with a tissue piercing distal tip, wherein the distal end releasably houses the glaucoma drainage device with at least part of the anchor or wire retained in a compressed state;
wherein at least part of the wire or anchor of the glaucoma drainage device is configured to automatically self-expand relative to the compressed state when deployed from the distal end of the rigid needle inserter to mechanically secure or fixate the glaucoma drainage device in the eye.

17. A glaucoma drainage system according to claim 16, wherein:
the rigid needle inserter includes a rod configured to push against the body or the wire or anchor of the glaucoma drainage device to deploy the glaucoma drainage device from the distal end of the rigid needle inserter.

18. A method of treating glaucoma of a human eye, comprising:
inserting a rigid needle inserter through ocular tissue to form a needle tract connected to an anterior chamber of the eye, wherein the needle inserter has a distal end with a tissue piercing distal tip, wherein the distal end releasably houses a glaucoma drainage device according to claim 1 with part of the expandable anchor or wire retained in a compressed state; and
deploying the glaucoma drainage device from the distal end of the needle inserter with the part of the anchor or wire disposed within the needle tract to permit the part of the expandable anchor or wire to automatically expand and mechanically secure or fixate the glaucoma drainage device in the needle tract.

19. A method according to claim 18, wherein:
the at least one expandable anchor or wire further comprises a sheath configured to expand radially with the part of the expandable anchor, and radial expansion of the sheath is configured to block peri-annular flow of aqueous humor around the body of the glaucoma drainage device.

20. A method according to claim 18, wherein:
the needle tract further connects to a drainage space to permit the glaucoma drainage device to shunt aqueous humor from the anterior chamber of the eye into the drainage space.

21. A method according to claim 20, wherein:
the drainage space is disposed under the conjunctiva and Tenon's capsule of the eye, or the drainage space is disposed in suprachoroidal space.

22. A method according to claim 18, wherein:
the rigid needle inserter includes a rod; and
deploying the glaucoma drainage device from the distal end of the needle inserter includes pushing the rod against the body or the wire or anchor of the glaucoma drainage device.
